(19) United States
(12) Patent Application Publication
(10) Pub. No.: US 2025/0161661 A1
(43) Pub. Date: May 22, 2025

(54) ELECTROPORATION PROBE WITH SELECTABLE INJECTION RATES AND SELECTABLE ELECTRIC FIELD STRENGTH

(71) Applicant: RadioClash, Ltd. Co., HOUSTON, TX (US)

(72) Inventors: Samit Dharia, Niskayuna, NY (US); Duane Bester, Dripping Springs, TX (US)

(21) Appl. No.: 18/515,326

(22) Filed: Nov. 21, 2023

(51) Int. Cl. A61N 1/04; A61N 1/20; A61N 1/32

(52) U.S. Cl. CPC A61N 1/0424; A61N 1/042; A61N 1/205; A61N 1/327

(57) ABSTRACT

An improved electroporation probe that delivers both electroporation and medicinal solution injection via a single instrument, and which allows altering of the applied electric field based on requirements of the target, is disclosed. A proximal end of a hollow tubular probe is connected to an injector for injecting medications, cells, proteins or biologics in solution into the probe, and which in turn are ejected through unsealed perforations on a perforated portion of the probe towards its distal end. A movable sleeve is slidable over the perforated portion of the probe in order to seal or unseal its perforations. A tapered metallic tip at the distal end of the probe is connected to an anode terminal of a battery, the distal end of the movable sleeve is connected to a cathode terminal of the battery. Separation between the tip and the distal end of the movable sleeve is varied to generate desired strength of the electric field needed for electroporation of target cells, or to selectively unseal more or fewer perforations for medicinal solution delivery at the target.

### FIELD OF THE INVENTION

The present invention relates to an electroporation probe that allows a single electroporation probe to be used for both the electroporation of cells and the injection of a therapeutic fluid.

### BACKGROUND

Electroporation based treatment is used for treating a variety of diseases including, but not limited to, various types of cancers. A current flowing through two or more probes inserted within a tumor region generates a voltage differential and corresponding field. Depending on the electrical field strength, permanent or temporary pores will be created within the cellular membranes of the tumor region in which the probes are inserted.

A higher electrical field strength applied by electroporation can render cell membrane pores to permanently open, leading to loss of homeostasis within the cells, and corresponding cell death. This type of electroporation is a type of non-thermal ablation called irreversible electroporation.

In reversible electroporation, a lower electrical field strength is applied such that cell membrane pores temporarily open (and hence become permeable for some time) and eventually close, leading to no loss of homeostasis, and no corresponding cell death. Reversible electroporation has been used in treatment of a variety of diseases coupled with delivery of medications and materials into the cells that are temporarily permeabilized.

Probes inserted into a tumor region and programmed to a high electrical field strength can cause both irreversible electroporation to occur in regions closer in proximity to the probes, and reversible electroporation to occur in regions further away from the probes, due to differences in electrical field strength in relation to the location of the probes. Combination electroporation therapy (CET) utilizes a high electrical field strength to cause irreversible electroporation to cells in closer proximity to the probes, while simultaneously injecting a medication or material into the tumor region to allow for increased cellular uptake in tumor regions further away from the probes that have had their cellular membranes temporarily permeabilized via the reversible electroporation effect. CET has been found to be more effective at inducing tumor cell death than either irreversible electroporation or intra-tumoral injection of chemotherapy alone, in various types of human cancers.

To perform CET, separate electroporation probes and injection needles are inserted into the patient to reach the target site. However, multiple equipment insertions or insertions at multiple sites lead to an increased potential for complications (including but not limited to bleeding, infection, and damage to adjacent structures) as well as increased discomfort for the patient, and further, can be difficult to perform.

U.S. Pat. No. 11,660,139 (incorporated herein by reference), addresses this problem by using a single probe for electroporation and injection of fluids. U.S. Pat. No. 11,660,139 discloses a hollow electroporation probe which is equipped to apply a desired electric field for electroporation of target cells, and which has a perforated distal end (i.e., the end which is inserted into the patient). The proximal end of the probe's tubing is attached to a fluid injector. Fluid injected into the hollow probe gets ejected through the perforations of the distal end. A longitudinally slideable internal sleeve lying within the hollow of the perforated distal end can be slid by an operator to seal or unseal the perforations at the distal end.

However, as the distal end is tapered, longitudinal sliding of the internal sleeve in and out of the tapered end can be difficult. Still further, since the distal end of the internal sleeve remains hidden within the probe, monitoring wear on the sleeve or the perforations in it is difficult. Additionally, better control over the strength the applied electric field based on the electroporation requirements of the target is also desired.

Therefore, there is a need for an improved device that delivers both electroporation and fluid injection via a single instrument, which allows altering of the applied electric field based on the requirements of the target, and enhances the sleeve life.

### SUMMARY

The present invention discloses an improved electroporation probe to be used for both the electroporation of cells and injection of a medicinal solution, and which provides better control over the strength the applied electric field based on electroporation requirements of the target. In one embodiment, the electroporation probe herein includes: a hollow tubular probe having a proximal end, a distal end and a plurality of perforations towards the distal end of the probe, said perforations connecting the hollow interior of said probe with its exterior, said proximal end being connected to a fluid injector and said distal end further including a tip;
a tubular movable sleeve covering at least the distal end of the probe, said sleeve being slidable longitudinally along the outer surface of the probe between a first position sealing each of said perforations and a second position unsealing each of said perforations, a distal end of said movable sleeve being connected to first terminal of the power source and said tip of said probe being connected to a second terminal of a power source for generation of an electroporation electric field between said tip and said distal end of said movable sleeve; and
a probe holder having an interior channel, where said tubular movable sleeve extends through the interior channel, and a sliding tab attached to the tubular sleeve such that longitudinal movement of the sliding tab moves the movable sleeve to either cover or uncover the perforations at the distal end of the probe.

Embodiments of the present invention will be discussed in greater details with reference to the accompanying figures in the detailed description which follows.

### BRIEF DESCRIPTION OF FIGURES

FIG. 1A illustrates a first embodiment of the electroporation probe, where the probe holder and the sliding tab are shown in cross-section. The sliding tab is shown in a released state, covering the probe perforations.

FIG. 1B illustrates the electroporation probe of FIG. 1A with the sliding tab moved to uncover the probe perforations.

FIG. 2A is an elevational view of portion ABCD of FIG. 1A.

FIG. 2B shows the portion in FIG. 2A but wherein the movable sleeve is shown in cross-section.

FIG. 2C is an elevational view of portion EFGH of FIG. 1B.

FIG. 2D shows the portion in FIG. 2C but wherein the movable sleeve and the probe tip are shown in cross-section.

FIGS. 3A and 3B are elevational views of the tip of the probe shown in FIGS. 2C and 2D.

FIG. 4 is a plan view of the tip of the probe shown in FIGS. 2C and 2D.

FIG. 5 illustrates a cross-section of tip of the probe shown in FIGS. 2C and 2D taken along plane RR′ of FIG. 4.

It should be understood that the drawings and the associated descriptions below are intended to illustrate one or more embodiments of the present invention, and not to limit the scope or the number of different possible embodiments of the invention.

The drawings are not necessarily drawn to scale.

### DETAILED DESCRIPTION

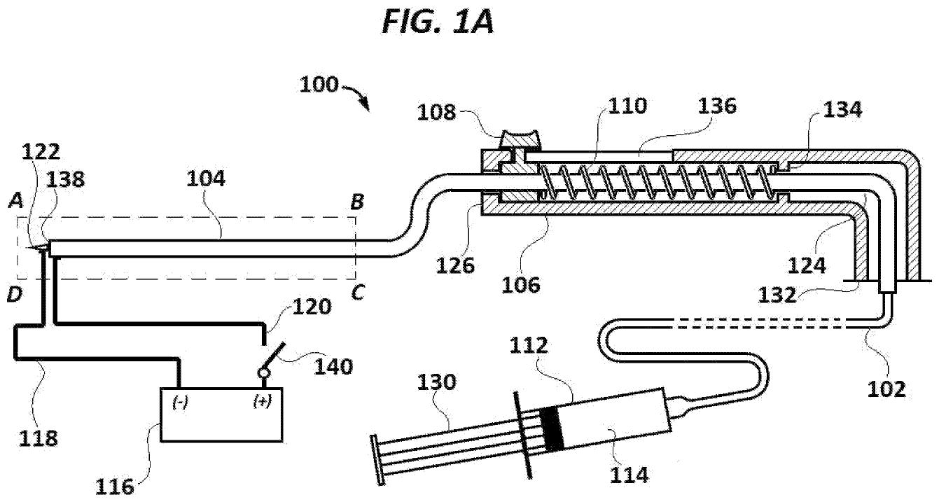

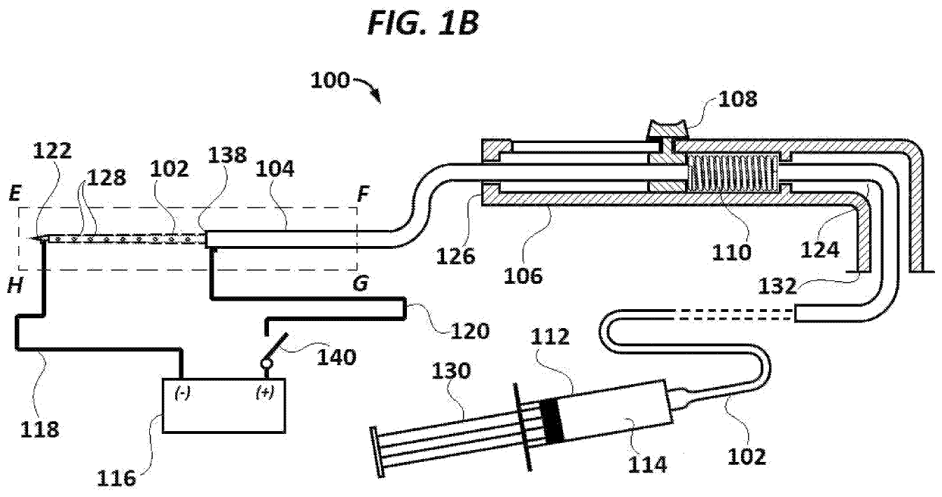

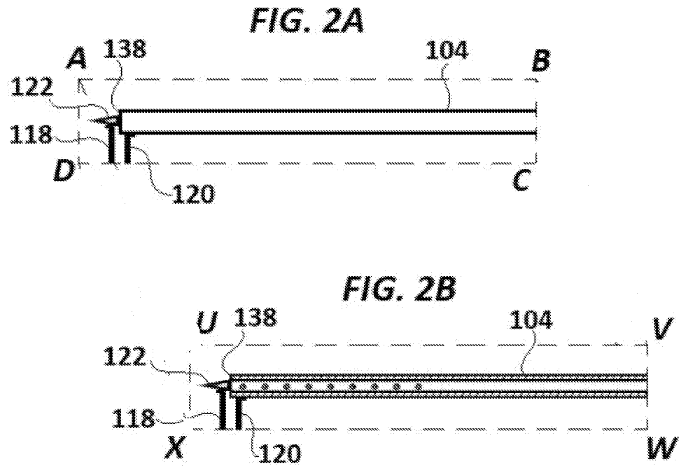

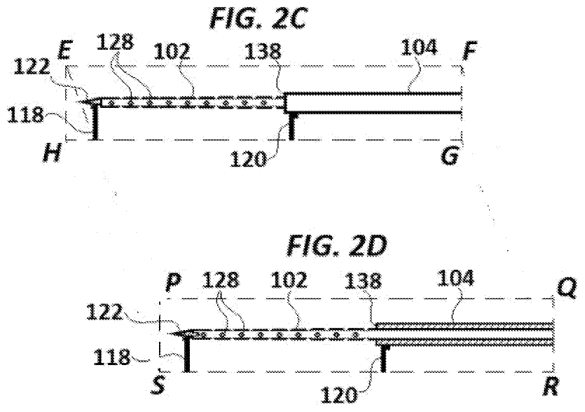

Reference will now be made in detail to a first embodiment of the electroporation probe of the present invention with reference to FIGS. 1A and 1B. As shown, the electroporation probe 100 incudes a flexible hollow tubular probe 102, a flexible tubular movable sleeve 104 covering a portion of length of probe 102, a probe holder 106, a sliding tab 108, a compression spring 110, a fluid injector 112, a power source 116, anode connection lead 118, and a cathode connection lead 120. The distal end of probe 102 includes a pointed tapered tip 122. A preset length of the probe 102, towards its distal end (near the tip 122), includes multiple perforations 128 (see FIGS. 1B and 2B-D). The proximal end of probe 102 is connected to the fluid injector 112 (such as a fluid syringe). The fluid injector 112 further includes a fluid reservoir 114 and a plunger 130.

When electroporation probe 100 is in resting state (as shown in FIG. 1A), probe 102 extends through a channel 124 of probe holder 106 and a substantial length of probe 102 from its distal end towards the proximal end is covered by movable sleeve 104. In this state, perforations 128 remain covered and sealed by movable sleeve 104. While distal end 138 of movable sleeve 104 remains flush with the distal end of probe 122 (and covers perforations 128), its proximal end extends into channel 124 of probe holder 106. Within probe holder 106, movable sleeve 104 (including probe 102 within) is surrounded by compression spring 110 (which is in an uncompressed state at rest).

Sliding tab 108 which is confined to slide within a longitudinal space 136 on the probe holder 106, is held against the distal end 126 of probe holder 106 by uncompressed spring 110. Movement of spring 110 towards proximal end 132 is restricted by an annular restriction 134 of the channel 124 within the probe holder 106. Sliding tab 108 is attached to movable sleeve 104. As a result, longitudinal movement of the sliding tab 108 moves movable sleeve 104 in the same direction.

Movement of sliding tab 108 away from the distal end 126 requires overcoming decompression force of the spring 110. On application of force by the user, sliding tab 108 (along with the movable sleeve 104 attached to it) is moved towards annular restriction 134, thereby compressing spring 110 (see FIG. 1B). As movable sleeve 104 slides along with sliding tab 108, its distal end 138 moves away from tip 122, to expose and unseal the perforated portion of probe 102. On removal of force on sliding tab 108, sliding tab 108 is pushed back towards distal end 126 of probe holder 106 by spring 110. Sliding tab 108 ends up at rest next to distal end 126, and movable sleeve 104 also moves to cover (and seal) perforations 128 of probe 102, and its distal end 138 ends up flush with tip 122.

The proximal end of tubular probe 102 is connected to fluid injector 112. When movable sleeve 104 is slid to expose (and unseal) perforations 128, any fluid carrying medication injected through the injector 112, travels through tubular probe 102, and gets ejected from perforations 128.

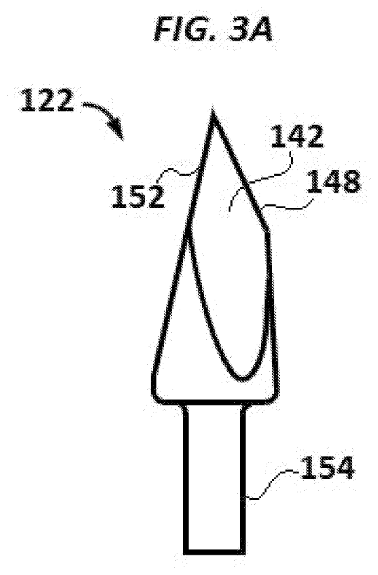

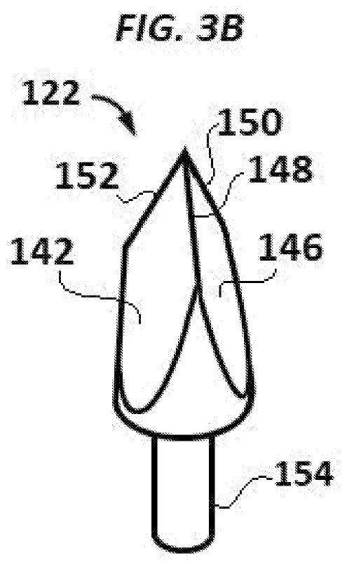

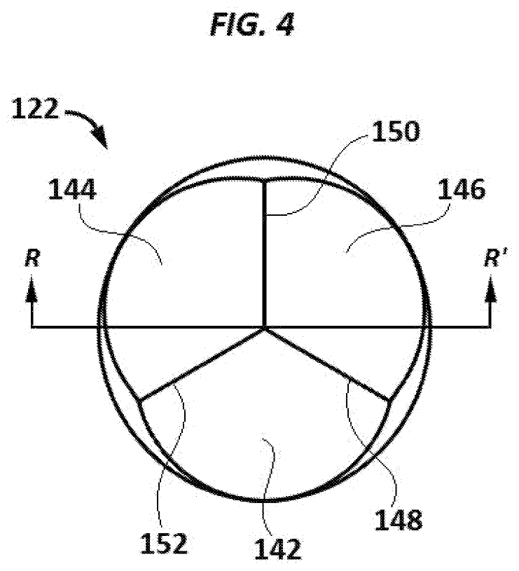

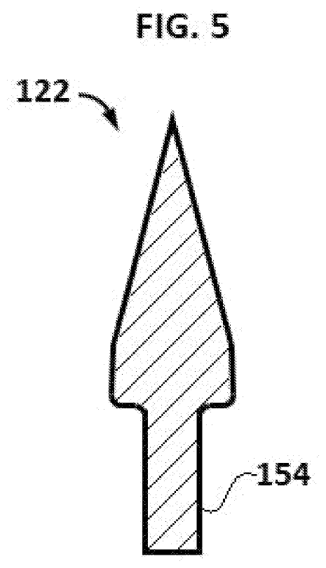

Tip 122 is metallic and its structure (as illustrated in FIGS. 3A-B, 4 and 5) is tapered and has a tri-planar surface. Its periphery includes three surrounding planes 142, 144 and 146. The interplanar edges 148, 150, and 152 are milled sharp for assisting the tip 122 to better push through tissues or cells. Tip 122 further includes a base shaft 154. Tip 122 is attached to the distal end of probe 106 by inserting the base shaft 154 into the hollow of probe 106.

The tip 122 of the probe 106 is connected to an anode terminal of a power source 116 (for example, a DC or re-chargeable battery) through a flexible and insulated anode connection lead 118. The distal end 138 of movable sleeve 104 is connected to the cathode terminal of the power source 116 through a flexible and insulated cathode connection lead 120, including a connection switch 140. Closing (or opening) of switch 140 results in connection (or disconnection) of distal end 138 of movable sleeve 104 with the power source 116.

It is to be understood that, in other embodiments of the invention, a similar switch may be provided in the anode connection lead 118 too for desirably connecting (or disconnecting) it with the power source 116. All such embodiments are fully covered within the scope of the present invention.

When Switch 140 is closed (i.e., in a circuit make position), an electric field is generated between the tip 122 and the distal end 138 of movable sleeve 104. The strength of the electric field depends on the amount of electric potential difference between the anode and cathode terminals of the power source and the amount of separation between the tip 122 and the distal end 138 of movable sleeve 104.

It is to be understood that the lengths of the probe 102 and the length movable sleeve 104 may be selected based on treatment requirements.

Though in the accompanying FIGS. 1A and 1B, the flexible and insulated anode connection lead 118 is illustrated as lying exterior to probe 102, in other embodiments of the invention it may lie within the hollow of probe 106 and remain being connected to tip 122 from within. Furthermore, in other embodiments, the flexible and insulated anode connection lead 118 may lie within the tubular body of probe 102 and remain connected to tip 122 from within.

Similarly, the flexible and insulated cathode connection lead 120 is illustrated as lying exterior to the movable sleeve 104, but in other embodiments it may lie within the hollow of movable sleeve 104 (in parallel with probe 102) and remain connected to distal end 138 at its periphery. In all such embodiments the presence of flexible and insulated cathode connection lead should not hinder smooth sliding of the movable sleeve 104 over probe 102. Still further, in other embodiments, the flexible and insulated cathode connection lead 118 may lie within the tubular body of movable sleeve 104 and remain connected to distal end 138 at its periphery.

Electroporation probe 100 is for treating target cells by delivering cells or proteins or biologics, or medications, which are in solution, with electroporation. For treating target cells or tissues, probe 106 is driven into the patient's body through an opening until tip 122 is placed at the target site. In the next step, depending on the amount of electroporation required, a desired strength of electric field is set between tip 122 and distal end 138 of movable sleeve 104. This is achieved by sliding the sliding tab 108 by a certain distance towards annular restriction 134, so that movable sleeve 104 is moved relative to tip 122 and is placed at a desired separation with the tip 122. Thereafter, switch 140 is closed to generate an electric field between tip 122 and distal end 138 of movable sleeve 104.

During or before the treatment, based on requirements, the strength of electric field may be controlled (i.e., increased or decreased) by maneuvering sliding tab 108 to control the separation between the tip 122 and the distal end 138 of the movable sleeve 104.

Medicinal solutions (including one or more of a medicinal drug and biological cells) can be delivered to the target site either in the presence or absence of applied electric field. When an electric field is applied, if the separation between the tip 122 and the distal end 138 of the movable sleeve 104 has sufficient perforations 128 exposed and unsealed, medications, cells or biologics can be delivered to the target site by injecting them into probe 102 using injector 112 (by pressing the plunger 130), until a desired quantity is ejected from the exposed perforations 128. However, if the number of exposed perforations 128 are insufficient to deliver the desired quantity, the separation between tip 122 and distal end 138 of the movable sleeve 104 can be increased to unseal more perforations 128. The applied electric field strength is reduced when the separation increases. The position may be changed after injection to again apply an increased strength electric field. Or, to deliver medications, cells or biologics to the target site in the absence of applied electric field, the switch 140 is turned off. Sliding tab 108 is moved to expose a desired length of the perforated portion of probe 106 before injection of medication, cells or other biologics.

It is to be understood that numerous variations and/or modifications may be made to the above-described embodiment, without departing from the scope of the present disclosure. The present embodiments are, therefore, to be considered in all respects as illustrative and not limiting.

What is claimed is:

1. An electroporation probe comprising:
a hollow tubular probe having a proximal end, a distal end and a plurality of perforations towards the distal end of the probe, said perforations connecting the hollow interior of said probe with its exterior, said proximal end being connected to a fluid injector and said distal end further including a tip;
a tubular movable sleeve covering at least a partial length of the probe, said sleeve being slidable longitudinally along the outer surface of the probe between a first position sealing each of said perforations and a second position unsealing each of said perforations, a distal end of said movable sleeve being connected to first terminal of a power source and said tip of said probe being connected to a second terminal of the power source for generation of an electroporation electric field between said tip and said distal end of said movable sleeve; and
a probe holder having an interior channel wherein said tubular movable sleeve extends through the interior channel, and a sliding tab attached to the tubular sleeve such that a longitudinal movement of the sliding tab away from the tip of the probe causes the movable sleeve to move from the first position towards the second position.

2. The electroporation probe as claimed in claim 1, wherein said power source is a DC battery.

3. The electroporation probe as claimed in claim 2, wherein said first terminal is a cathode terminal of said DC battery and the second terminal is an anode terminal of said DC battery.

4. The electroporation probe as claimed in claim 1, wherein said tip is metallic and tapered.

5. The electroporation probe as claimed in claim 1, wherein the hollow tubular probe and the tubular movable sleeve are flexible.

6. The electroporation probe as claimed in claim 1, wherein movement of said sliding tab away from the tip of said probe is resisted by a compression spring.

7. The electroporation probe as claimed in claim 1, wherein the compression spring is included in said probe holder.

8. The electroporation probe as claimed in claim 1, wherein said distal end of said movable sleeve is connected to first terminal of the power source through a switch

9. The electroporation probe as claimed in claim 1, wherein said tip of said probe is connected to the second terminal of the power source through a switch.

10. A method of providing electroporation and a medicinal solution at a target site, said method comprising:
placing a tip, said tip being at a distal end of a hollow tubular probe, and a distal end of a tubular movable sleeve covering at least a partial length of said probe on a target site, said hollow tubular probe having a proximal end, and a plurality of perforations towards the distal end, said perforations connecting the hollow interior of said probe with its exterior, said proximal end being connected to a fluid injector, said tubular movable sleeve being slidable longitudinally along the outer surface of the probe between a first position sealing each of said perforations and a second position unsealing each of said perforations;
achieving a desired separation between said distal end of said movable sleeve and said tip by sliding said movable sleeve using a sliding tab attached to the tubular sleeve such that a longitudinal movement of the sliding tab away from the tip of the probe causes said movable sleeve to slide from the first position towards the second position, said sliding tab being included in a probe holder having an interior channel wherein said tubular movable sleeve extends through the interior channel; and
performing at least one of the following steps:
i) enabling an electrical connection between a first electrical conductor lead connected to said distal end of said movable sleeve with a first terminal of a power source and, enabling an electrical connection between a second electrical conductor lead connected to said tip of said probe with a second terminal of the power source for generation of an electroporation electric field between said tip and said distal end of said movable sleeve; and
ii) delivering a medicinal solution to the target site by injecting said medicinal solution into the probe, for being ejected from unsealed perforations on the probe, through the fluid injector.

11. The method as claimed in claim 10, wherein said power source is a DC battery.

12. The method as claimed in claim 11, wherein said first terminal is a cathode terminal of said DC battery and the second terminal is an anode terminal of said DC battery.

13. The method as claimed in claim 10, wherein said tip is metallic and tapered.

14. The method as claimed in claim 10, wherein the hollow tubular probe and the tubular movable sleeve are flexible.

15. The method as claimed in claim 10, wherein movement of said sliding tab away from the tip of said probe is resisted by a compression spring.

16. The method as claimed in claim 10, wherein the compression spring is included in said probe holder.

17. The method as claimed in claim 10, wherein said distal end of said movable sleeve is connected to first terminal of the power source through a switch.

18. The method as claimed in claim 10, wherein said tip of said probe is connected to the second terminal of the power source through a switch.

19. The method as claimed in claim 10, wherein said medicinal solution includes one or more of a drug, antibodies, protein and cells.
